(19) United States
(12) Patent Application Publication
(10) Pub. No.: US 2018/0333089 A1
(43) Pub. Date: Nov. 22, 2018

(54) METHOD, SYSTEMS, AND KITS USING REDUCED OXYGEN FOR PHYSIOLOGICAL BENEFIT

(71) Applicant: Richard Dale Rife, III, Greenwood, IN (US)

(72) Inventor: Richard Dale Rife, III, Greenwood, IN (US)

(21) Appl. No.: 15/795,213

(22) Filed: Oct. 26, 2017

Related U.S. Application Data
(60) Provisional application No. 62/603,076, filed on May 16, 2017.

(51) Int. Cl. A61B 5/1455; A61B 5/145; A61B 5/024

(52) U.S. Cl. CPC A61B 5/14552; A61B 5/6813; A61B 5/02427; A61B 5/14542

(57) ABSTRACT

Methods, systems, and kits are disclosed for facilitating physiological benefit in using activities with a reduced oxygen as an added catalyst during inactive periods. Substantial physiological benefits exist in the realm of muscular force output whereby reduced oxygen is inspired by a subject during the inactive periods of activities.

### CROSS-REFERENCE TO RELATED APPLICATIONS

This application claims the benefit of provisional patent application Ser. No. 62/603,076 filed 2017 May 16 by the present inventor.

### FEDERALLY SPONSORED RESEARCH

None.

### SEQUENCE LISTING

None.

### BACKGROUND

Conventional exercise is based on strength and cardiovascular training activities to enhance movement, physical strength, and overall health. Where limitations in cardiovascular and muscular strength are an issue, exercise activities, biological agents, and/or supplements to improve previous and current states are recommended. Conventional exercise additives may overload physiology leading to degradation and/or injury. Conventional biological agents and/or supplements may cause undesired side effects to the subject despite the desired benefits.

The strategies for muscular strength methods, systems, and kits are continually researched and need multiple applicable solutions. Perspectives have also been developed on the principle of human exercise being fundamental to health and the loss of muscular force output may be shown as a decrease in physical performance and recovery.

There exists physiological benefits as a subject adapts to an acute hypoxic condition. The recent ability to simulate a reduced oxygen environment allows the subject to introduce these benefits to cardiovascular and/or strength training exercise activities for its beneficial outcomes with reduced risk and known side effects compared to other methods.

Cardiovascular exercise in hypoxia or living in hypoxia and cardiovascular training in normoxia can be used to drive a subject's productive and efficient endurance. Strength training in hypoxia can be used to drive a subject's hypertrophy and anabolic state for muscular growth and development.

Hypoxic strength training uses two known methods to create an internal change. One uses a pressurized external barrier around tissue, known as vascular occlusion or blood flow restriction. The other uses a device attached to a hosed mask or a room delivering a reduced oxygen air during exercise. Vascular occlusion adds different devices to the body leading to limits in possible exercise activities. The mask version creates the risk of having the attached hose inhibit an exercise activity and the constant limit of available oxygen for a subject's recovery from intense exercise activities. Performing exercise activities within a hypoxic room challenges the intensity of available exercise activities and decrease anabolic benefit due to the constant stress of the hypoxic state on a subject.

Within this document are disclosures and embodiments described as novel solutions to improving muscular force output while using an acute hypoxic condition from a reduced oxygen environment.

The present method is directed toward overcoming one or more of the problems discussed above.

### SUMMARY

This document provides methods, systems and kits for physiological benefit through activities with the addition of a reduced oxygen environment during inactive periods prior to and following each activity. The methods, systems, and kits are designed to enhance the subject's physiology as they relate to muscular force output while performing an activity. The methods and systems described in this document provide for significant improvements in muscular force output over conventional exercise methods as shown in FIG. 1.

Also, this document includes methods and systems for assessing and introducing a reduced oxygen environment as an initial episode and subsequent episodes during the inactive period between activities.

This method separates itself from others by introducing and limiting the reduced oxygen environment to rest periods or inactive periods exclusively.

The disclosure also provides kits for facilitating the methods and systems.

### DRAWINGS

FIG. 1—Muscular force output overtime comparison between the acute hypoxic at rest method versus the convention normoxic method

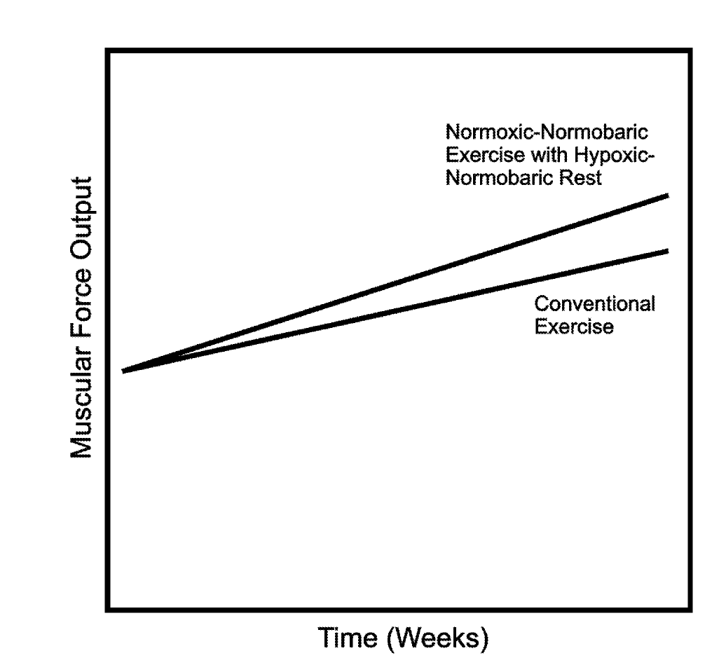

### DETAILED DESCRIPTION

In more detail, inspiring reduced oxygen air, using the methods, systems and kits, initially causes a subject's blood oxygen saturation to be reduced to a predetermined level, thereby creating a biological and physiological environment suitable for improving muscular force output as long as the addition of an external force is provided immediately following the hypoxic episode. Once the subject is maintaining a blood oxygen saturation below a predetermined level (but not lower than 80%), then following the activity causes a subject's chemoreflex to respond with acute adaptations to the external load. After a sufficient amount of time in the blood oxygen saturation zone of a predetermined level down to 80%, herein known as the BOS zone, the subject begins the chosen activity. The activity period is immediately followed by reintroduction to the hypoxic-normobaric environment until the BOS zone is reach for a sufficient amount of time. The cycle is repeated before and after for all future applications of activities.

In addition, a subject using these methods, systems and kits described in this document offers an internal hypoxic environment while performing activities without external obstruction during movement, such as vascular occlusion, and without symptoms related to altitude sickness, such as training with simultaneously inspiration of reduced oxygen.

Operation

Generally, methods disclosed in this document provide for the introduction of a reduced oxygen environment, and thereby the catalyst, to stimulate a physiological response conducive to muscular growth and development when coupled with activities. In addition, the method necessitates the requirement for a kit to deliver the reduced oxygen content, a kit to monitor the percentage of oxygen from the oxygen content-reducing device, a kit to monitor a subject's blood oxygen saturation, and kits related to activities performed by the subject.

Embodiments in this document provide the order of action for the methods, systems and kits. Generally, the methods consist of recording a subject's normoxic and normobaric baseline assessment, recording a subject's hypoxic and normobaric baseline assessment, and recording a subject's hypoxic and normobaric activity session. As such, the assessments are necessary to determine physiological changes or anomalies of a subject needing to be addressed outside of this method.

### 1. Normobaric-Normoxic Baseline Assessment

### 2. Normobaric-Hypoxic Baseline Assessment

### 3. Normobaric-Hypoxic Activity Session

a. Assess Current State
b. Reduced Oxygen Environment during Inactive or Rest period
c. BOS Zone for a sufficient amount of time
d. Normal Oxygen Environment during Activity
e. Assess Current State
f. Repeat b, c, d, e until Activity Session is complete
4. Repeat 3 until activity plan is complete

The normoxic and normobaric baseline assessment begins with recording a subject's blood oxygen saturation measurement using a pulse oximeter before and after each resting period. The pulse oximeter should be on the subject's finger to begin measurement of their blood oxygen saturation. Following the recording of the data, a subject proceeds to activities as necessary to achieve desired outcomes. Between each activity, the subject's blood oxygen saturation measurement is recorded after completion and monitored for a sufficient amount of time. This cycle is repeated for all activities. Upon completion of all activities, the subject's blood oxygen saturation measurement is recorded and monitored for a final time. If there are not any anomalies with the subject's activities, then the subject returns at a later date to assess their response to a reduced oxygen environment.

At a later date, the hypoxic and normobaric baseline assessment begins with a subject's blood oxygen saturation measurement using a pulse oximeter prior to the reduced oxygen environment introduction. Following the recording of the initial data, the method proceeds to the introduction of a subject to the reduced oxygen environment. The initial setting of FiO2 (fraction of inspired oxygen) on the oxygen reducing device is sufficient to begin induction of hypoxia The pulse oximeter is placed on the subject's finger to begin measurement of their blood oxygen saturation. When a predetermined level is reached, the subject continues breathing in the reduced oxygen environment for a sufficient amount of time below a predetermined level, but not lower than 80%. The blood oxygen saturation percentage between a predetermined level and 80% is also known as the BOS zone. If the subject's blood oxygen saturation reading does not reach the BOS zone within a sufficient amount of time, then reduce the FiO2 in 1% increments until the BOS zone is reached. The final FiO2 reading is the future starting setting for this subject. Immediately following being in the BOS zone for a sufficient amount of time, the subject begins the first activity as necessary to achieve desired outcomes. Immediately following each activity, the subject is reintroduced to their FiO2 setting and the blood oxygen saturation. The subject should return to the BOS zone within a sufficient amount of time depending on the subject's condition and/or the intensity of the activity. The subject remains in the BOS zone for a sufficient amount of time and returns immediately to the predetermined activity. The cycle should be repeated until all the predetermined activities are complete. Upon completion of all activities, the subject returns to the reduced oxygen environment for the final BOS zone while recording and monitoring the blood oxygen saturation. If anomalies do not exist at any time during these cycles as shown below, then the subject can continue.

A subject can repeat the following cycle in all future activity sessions. Record the blood oxygen saturation measurement of the subject using a pulse oximeter on the subject prior to hypoxic introduction. Following the recording of the initial data, the subject proceeds to the introduction of the reduced oxygen environment. The final setting of FiO2 from the hypoxic-normobaric baseline assessment or the previous activity session is the subject's FiO2 setting, but may need to be adjusted during the series of activities depending on the subject's condition, the activity intensity, or any other factors specific to the subject. When the BOS Zone is reached, then the subject continues breathing the reduced oxygen air for a sufficient amount of time. If the subject's blood oxygen saturation reading does not reach the BOS zone within a sufficient amount of time again, then reduce the FiO2 in 1% increments until at least 93% is reached. Immediately following being in the BOS zone for a sufficient amount of time, the subject begins the first activity as necessary to achieve desired outcomes. Immediately following each activity, the subject is reintroduced to the subject's FiO2 setting and the blood oxygen saturation is recorded and monitored from the pulse oximeter. The subject should return to the BOS zone within a sufficient amount of time, then remain in the BOS zone for a sufficient amount of time and return to the next activity. This cycle should be repeated until all activities are complete. Upon completion of activities, the subject returns to the reduced oxygen environment for the final BOS zone time period while recording and monitoring the blood oxygen saturation. Again if anomalies do not exist at any time during the session, then the cycle can continue.

### EXAMPLES

### Example 1

Step 1: Assess subject's physiological state including but not limited to heart rate, blood pressure, blood oxygen saturation and current mood.

Step 2A: Introduce the subject to a reduced oxygen environment via a mask delivered from a hypoxic generator and continue to monitor the blood oxygen saturation and heart rate.

Step 2B: Reduce and maintain the subject's blood oxygen saturation below 93%, but above 80% for at least 60 seconds to elicit the desired response for the upcoming squat strength training exercise.

Step 2C: The subject must immediately begin the squat strength training exercise.

Step 2D: Assess the subject physiological state including, but not limited to, heart rate, blood oxygen saturation and current mood.

Step 3A: Reintroduce the subject to a reduced oxygen environment via a mask delivered from a hypoxic generator and continue to monitor the blood oxygen saturation and heart rate.

Step 3B: Reduce and maintain the subject's blood oxygen saturation below 93%, but above 80% for at least 60 seconds to elicit the desired response for the upcoming squat strength training exercise.

Step 3C: The subject must immediately begin the squat strength training exercise.

Step 3D: Assess the subject physiological state including, but not limited to, heart rate, blood oxygen saturation and current mood.

Step 4A: Reintroduce the subject to a reduced oxygen environment via a mask delivered from a hypoxic generator and continue to monitor the blood oxygen saturation and heart rate.

Step 4B: Reduce and maintain the subject's blood oxygen saturation below 93%, but above 80% for at least 60 seconds to elicit the desired response for the upcoming squat strength training exercise.

Step 4C: The subject must immediately begin the squat strength training exercise.

Step 4D: Assess the subject physiological state including, but not limited to, heart rate, blood oxygen saturation and current mood.

Step 5A: Reintroduce the subject to a reduced oxygen environment via a mask delivered from a hypoxic generator and continue to monitor the blood oxygen saturation and heart rate.

Step 5B: Reduce and maintain the subject's blood oxygen saturation below 93%, but above 80% for at least 60 seconds to elicit the desired response for the upcoming chest press strength training exercise.

Step 5C: The subject must immediately begin the chest press strength training exercise.

Step 5D: Assess the subject physiological state including, but not limited to, heart rate, blood oxygen saturation and current mood.

Step 6A: Reintroduce the subject to a reduced oxygen environment via a mask delivered from a hypoxic generator and continue to monitor the blood oxygen saturation and heart rate.

Step 6B: Reduce and maintain the subject's blood oxygen saturation below 93%, but above 80% for at least 60 seconds to elicit the desired response for the upcoming chest press strength training exercise.

Step 6C: The subject must immediately begin the chest press strength training exercise.

Step 6D: Assess the subject physiological state including, but not limited to, heart rate, blood oxygen saturation and current mood.

Step 7A: Reintroduce the subject to a reduced oxygen environment via a mask delivered from a hypoxic generator and continue to monitor the blood oxygen saturation and heart rate.

Step 7B: Reduce and maintain the subject's blood oxygen saturation below 93%, but above 80% for at least 60 seconds to elicit the desired response for the upcoming chest press strength training exercise.

Step 7C: The subject must immediately begin the chest press strength training exercise.

Step 7D: Assess the subject physiological state including, but not limited to, heart rate, blood oxygen saturation and current mood.

Step 8A: Reintroduce the subject to a reduced oxygen environment via a mask delivered from a hypoxic generator and continue to monitor the blood oxygen saturation and heart rate.

Step 8B: Reduce and maintain the subject's blood oxygen saturation below 93%, but above 80% for at least 60 seconds to elicit the desired response for the upcoming row strength training exercise.

Step 8C: The subject must immediately begin the row strength training exercise.

Step 8D: Assess the subject physiological state including, but not limited to, heart rate, blood oxygen saturation and current mood.

Step 9A: Reintroduce the subject to a reduced oxygen environment via a mask delivered from a hypoxic generator and continue to monitor the blood oxygen saturation and heart rate.

Step 9B: Reduce and maintain the subject's blood oxygen saturation below 93%, but above 80% for at least 60 seconds to elicit the desired response for the upcoming row strength training exercise.

Step 9C: The subject must immediately begin the row strength training exercise.

Step 9D: Assess the subject physiological state including, but not limited to, heart rate, blood oxygen saturation and current mood.

Step 10A: Reintroduce the subject to a reduced oxygen environment via a mask delivered from a hypoxic generator and continue to monitor the blood oxygen saturation and heart rate.

Step 10B: Reduce and maintain the subject's blood oxygen saturation below 93%, but above 80% for at least 60 seconds to elicit the desired response for the upcoming row strength training exercise.

Step 10C: The subject must immediately begin the row strength training exercise.

Step 10D: Assess the subject physiological state including, but not limited to, heart rate, blood oxygen saturation and current mood.

Step 11A: Reintroduce the subject to a reduced oxygen environment via a mask delivered from a hypoxic generator and continue to monitor the blood oxygen saturation and heart rate.

Step 11B: Reduce and maintain the subject's blood oxygen saturation below 93%, but above 80% for at least 60 seconds to elicit the desired response for the upcoming shoulder press strength training exercise.

Step 11C: The subject must immediately begin the shoulder press strength training exercise.

Step 11D: Assess the subject physiological state including, but not limited to, heart rate, blood oxygen saturation and current mood.

Step 12A: Reintroduce the subject to a reduced oxygen environment via a mask delivered from a hypoxic generator and continue to monitor the blood oxygen saturation and heart rate.

Step 12B: Reduce and maintain the subject's blood oxygen saturation below 93%, but above 80% for at least 60 seconds to elicit the desired response for the upcoming shoulder press strength training exercise.

Step 12C: The subject must immediately begin the shoulder press strength training exercise.

Step 12D: Assess the subject physiological state including, but not limited to, heart rate, blood oxygen saturation and current mood.

Step 13A: Reintroduce the subject to a reduced oxygen environment via a mask delivered from a hypoxic generator and continue to monitor the blood oxygen saturation and heart rate.

Step 13B: Reduce and maintain the subject's blood oxygen saturation below 93%, but above 80% for at least 60 seconds to elicit the desired response for the upcoming shoulder press strength training exercise.

Step 13C: The subject must immediately begin the shoulder press strength training exercise.

Step 13D: Assess the subject physiological state including, but not limited to, heart rate, blood oxygen saturation and current mood.

Step 14A: Reintroduce the subject to a reduced oxygen environment via a mask delivered from a hypoxic generator and continue to monitor the blood oxygen saturation and heart rate.

Step 14B: Reduce and maintain the subject's blood oxygen saturation below 93%, but above 80% for at least 60 seconds to elicit the desired response for the upcoming lat pulldown strength training exercise.

Step 14C: The subject must immediately begin the lat pulldown strength training exercise.

Step 14D: Assess the subject physiological state including, but not limited to, heart rate, blood oxygen saturation and current mood.

Step 15A: Reintroduce the subject to a reduced oxygen environment via a mask delivered from a hypoxic generator and continue to monitor the blood oxygen saturation and heart rate.

Step 15B: Reduce and maintain the subject's blood oxygen saturation below 93%, but above 80% for at least 60 seconds to elicit the desired response for the upcoming lat pulldown strength training exercise.

Step 15C: The subject must immediately begin the lat pulldown strength training exercise.

Step 15D: Assess the subject physiological state including, but not limited to, heart rate, blood oxygen saturation and current mood.

Step 16A: Reintroduce the subject to a reduced oxygen environment via a mask delivered from a hypoxic generator and continue to monitor the blood oxygen saturation and heart rate.

Step 17B: Reduce and maintain the subject's blood oxygen saturation below 93%, but above 80% for at least 60 seconds to elicit the desired response for the upcoming lat pulldown strength training exercise.

Step 17C: The subject must immediately begin the lat pulldown strength training exercise.

Step 17D: Assess the subject physiological state including, but not limited to, heart rate, blood oxygen saturation and current mood.

Step 18A: Reintroduce the subject to a reduced oxygen environment via a mask delivered from a hypoxic generator and continue to monitor the blood oxygen saturation and heart rate.

Step 18B: Reduce and maintain the subject's blood oxygen saturation below 93%, but above 80% for at least 60 seconds to elicit the desired response for the upcoming abdominal strength training exercise.

Step 18C: The subject must immediately begin the abdominal strength training exercise.

Step 18D: Assess the subject physiological state including, but not limited to, heart rate, blood oxygen saturation and current mood.

Step 19A: Reintroduce the subject to a reduced oxygen environment via a mask delivered from a hypoxic generator and continue to monitor the blood oxygen saturation and heart rate.

Step 19B: Reduce and maintain the subject's blood oxygen saturation below 93%, but above 80% for at least 60 seconds to elicit the desired response for the upcoming abdominal strength training exercise.

Step 19C: The subject must immediately begin the abdominal strength training exercise.

Step 19D: Assess the subject physiological state including, but not limited to, heart rate, blood oxygen saturation and current mood.

Step 20A: Reintroduce the subject to a reduced oxygen environment via a mask delivered from a hypoxic generator and continue to monitor the blood oxygen saturation and heart rate.

Step 20B: Reduce and maintain the subject's blood oxygen saturation below 93%, but above 80% for at least 60 seconds to elicit the desired response for the upcoming abdominal strength training exercise.

Step 20C: The subject must immediately begin the abdominal strength training exercise.

Step 20D: Assess the subject physiological state including, but not limited to, heart rate, blood oxygen saturation and current mood.

Step 21: Reintroduce the subject to a reduced oxygen environment via a mask delivered from a hypoxic generator and continue to monitor the blood oxygen saturation and heart rate.

Step 22: Reduce and maintain the subject's blood oxygen saturation below 93%, but above 80% for at least 60 seconds to elicit the desired response.

Step 23: Refrain from inspiring reduced oxygen until the next exercise session.

### Example 2

Step 1: Assess subject's physiological state including but not limited to heart rate, blood pressure, blood oxygen saturation and current mood.

Step 2A: Introduce the subject to a reduced oxygen environment via a mask delivered from a hypoxic generator and continue to monitor the blood oxygen saturation and heart rate.

Step 2B: Reduce and maintain the subject's blood oxygen saturation below 93%, but above 80% for at least 60 seconds to elicit the desired response for the upcoming jogging exercise.

Step 2C: The subject must immediately begin the jogging exercise at a specified intensity for one to five minutes depending on the desired outcome.

Step 2D: Assess the subject physiological state including, but not limited to, heart rate, blood oxygen saturation and current mood.

Step 3A: Reintroduce the subject to a reduced oxygen environment via a mask delivered from a hypoxic generator and continue to monitor the blood oxygen saturation and heart rate.

Step 3B: Reduce and maintain the subject's blood oxygen saturation below 93%, but above 80% for at least 60 seconds to elicit the desired response for the upcoming cardiovascular training exercise.

Step 3C: The subject must immediately begin the cardiovascular training exercise at a specified intensity for one to five minutes depending on the desired outcome and the recovery from the previous exercise.

Step 3D: Assess the subject physiological state including, but not limited to, heart rate, blood oxygen saturation and current mood.

Step 4A: Reintroduce the subject to a reduced oxygen environment via a mask delivered from a hypoxic generator and continue to monitor the blood oxygen saturation and heart rate.

Step 4B: Reduce and maintain the subject's blood oxygen saturation below 93%, but above 80% for at least 60 seconds to elicit the desired response for the upcoming jogging exercise.

Step 4C: The subject must immediately begin the jogging exercise at a specified intensity for one to five minutes depending on the desired outcome and the recovery from the previous exercise.

Step 4D: Assess the subject physiological state including, but not limited to, heart rate, blood oxygen saturation and current mood.

Step 5A: Reintroduce the subject to a reduced oxygen environment via a mask delivered from a hypoxic generator and continue to monitor the blood oxygen saturation and heart rate.

Step 5B: Reduce and maintain the subject's blood oxygen saturation below 93%, but above 80% for at least 60 seconds to elicit the desired response for the upcoming jogging exercise.

Step 5C: The subject must immediately begin the jogging exercise at a specified intensity for one to five minutes depending on the desired outcome and the recovery from the previous exercise.

Step 5D: Assess the subject physiological state including, but not limited to, heart rate, blood oxygen saturation and current mood.

Step 6A: Reintroduce the subject to a reduced oxygen environment via a mask delivered from a hypoxic generator and continue to monitor the blood oxygen saturation and heart rate.

Step 6B: Reduce and maintain the subject's blood oxygen saturation below 93%, but above 80% for at least 60 seconds to elicit the desired response for the upcoming cycling exercise.

Step 6C: The subject must immediately begin the cycling exercise at a specified intensity for one to five minutes depending on the desired outcome and the recovery from the previous exercise.

Step 6D: Assess the subject physiological state including, but not limited to, heart rate, blood oxygen saturation and current mood.

Step 7A: Reintroduce the subject to a reduced oxygen environment via a mask delivered from a hypoxic generator and continue to monitor the blood oxygen saturation and heart rate.

Step 7B: Reduce and maintain the subject's blood oxygen saturation below 93%, but above 80% for at least 60 seconds to elicit the desired response for the upcoming cycling exercise.

Step 7C: The subject must immediately begin the cycling exercise at a specified intensity for one to five minutes depending on the desired outcome and the recovery from the previous exercise.

Step 7D: Assess the subject physiological state including, but not limited to, heart rate, blood oxygen saturation and current mood.

Step 8A: Reintroduce the subject to a reduced oxygen environment via a mask delivered from a hypoxic generator and continue to monitor the blood oxygen saturation and heart rate.

Step 8B: Reduce and maintain the subject's blood oxygen saturation below 93%, but above 80% for at least 60 seconds to elicit the desired response for the upcoming cycling exercise.

Step 8C: The subject must immediately begin the cycling exercise at a specified intensity for one to five minutes depending on the desired outcome and the recovery from the previous exercise.

Step 8D: Assess the subject physiological state including, but not limited to, heart rate, blood oxygen saturation and current mood.

Step 9A: Reintroduce the subject to a reduced oxygen environment via a mask delivered from a hypoxic generator and continue to monitor the blood oxygen saturation and heart rate.

Step 9B: Reduce and maintain the subject's blood oxygen saturation below 93%, but above 80% for at least 60 seconds to elicit the desired response for the upcoming rowing exercise.

Step 9C: The subject must immediately begin the rowing exercise at a specified intensity for one to five minutes depending on the desired outcome and the recovery from the previous exercise.

Step 9D: Assess the subject physiological state including, but not limited to, heart rate, blood oxygen saturation and current mood.

Step 10A: Reintroduce the subject to a reduced oxygen environment via a mask delivered from a hypoxic generator and continue to monitor the blood oxygen saturation and heart rate.

Step 10B: Reduce and maintain the subject's blood oxygen saturation below 93%, but above 80% for at least 60 seconds to elicit the desired response for the upcoming cycling exercise.

Step 10C: The subject must immediately begin the cycling exercise at a specified intensity for one to five minutes depending on the desired outcome and the recovery from the previous exercise.

Step 10D: Assess the subject physiological state including, but not limited to, heart rate, blood oxygen saturation and current mood.

Step 11A: Reintroduce the subject to a reduced oxygen environment via a mask delivered from a hypoxic generator and continue to monitor the blood oxygen saturation and heart rate.

Step 11B: Reduce and maintain the subject's blood oxygen saturation below 93%, but above 80% for at least 60 seconds to elicit the desired response for the upcoming cycling exercise.

Step 11C: The subject must immediately begin the cycling exercise at a specified intensity for one to five minutes depending on the desired outcome and the recovery from the previous exercise.

Step 11D: Assess the subject physiological state including, but not limited to, heart rate, blood oxygen saturation and current mood.

Step 12A: Reintroduce the subject to a reduced oxygen environment via a mask delivered from a hypoxic generator and continue to monitor the blood oxygen saturation and heart rate.

Step 12B: Reduce and maintain the subject's blood oxygen saturation below 93%, but above 80% for at least 60 seconds to elicit the desired response for the upcoming rowing exercise.

Step 12C: The subject must immediately begin the rowing exercise at a specified intensity for one to five minutes depending on the desired outcome and the recovery from the previous exercise.

Step 12D: Assess the subject physiological state including, but not limited to, heart rate, blood oxygen saturation and current mood.

Step 13A: Reintroduce the subject to a reduced oxygen environment via a mask delivered from a hypoxic generator and continue to monitor the blood oxygen saturation and heart rate.

Step 13B: Reduce and maintain the subject's blood oxygen saturation below 93%, but above 80% for at least 60 seconds to elicit the desired response for the upcoming rowing exercise.

Step 13C: The subject must immediately begin the rowing exercise at a specified intensity for one to five minutes depending on the desired outcome and the recovery from the previous exercise.

Step 13D: Assess the subject physiological state including, but not limited to, heart rate, blood oxygen saturation and current mood.

Step 14A: Reintroduce the subject to a reduced oxygen environment via a mask delivered from a hypoxic generator and continue to monitor the blood oxygen saturation and heart rate.

Step 14B: Reduce and maintain the subject's blood oxygen saturation below 93%, but above 80% for at least 60 seconds to elicit the desired response for the upcoming rowing exercise.

Step 14C: The subject must immediately begin the rowing exercise at a specified intensity for one to five minutes depending on the desired outcome and the recovery from the previous exercise.

Step 14D: Assess the subject physiological state including, but not limited to, heart rate, blood oxygen saturation and current mood.

Step 15A: Reintroduce the subject to a reduced oxygen environment via a mask delivered from a hypoxic generator and continue to monitor the blood oxygen saturation and heart rate.

Step 15B: Reduce and maintain the subject's blood oxygen saturation below 93%, but above 80% for at least 60 seconds to elicit the desired response for the upcoming running exercise.

Step 15C: The subject must immediately begin the running exercise at a specified intensity for one to five minutes depending on the desired outcome and the recovery from the previous exercise.

Step 15D: Assess the subject physiological state including, but not limited to, heart rate, blood oxygen saturation and current mood.

Step 16A: Reintroduce the subject to a reduced oxygen environment via a mask delivered from a hypoxic generator and continue to monitor the blood oxygen saturation and heart rate.

Step 17B: Reduce and maintain the subject's blood oxygen saturation below 93%, but above 80% for at least 60 seconds to elicit the desired response for the upcoming running exercise.

Step 17C: The subject must immediately begin the running exercise at a specified intensity for one to five minutes depending on the desired outcome and the recovery from the previous exercise.

Step 17D: Assess the subject physiological state including, but not limited to, heart rate, blood oxygen saturation and current mood.

Step 18A: Reintroduce the subject to a reduced oxygen environment via a mask delivered from a hypoxic generator and continue to monitor the blood oxygen saturation and heart rate.

Step 18B: Reduce and maintain the subject's blood oxygen saturation below 93%, but above 80% for at least 60 seconds to elicit the desired response for the upcoming walking exercise.

Step 18C: The subject must immediately begin the walking exercise at a specified intensity for one to five minutes depending on the desired outcome and the recovery from the previous exercise.

Step 18D: Assess the subject physiological state including, but not limited to, heart rate, blood oxygen saturation and current mood.

Step 19A: Reintroduce the subject to a reduced oxygen environment via a mask delivered from a hypoxic generator and continue to monitor the blood oxygen saturation and heart rate.

Step 19B: Reduce and maintain the subject's blood oxygen saturation below 93%, but above 80% for at least 60 seconds to elicit the desired response for the upcoming walking exercise.

Step 19C: The subject must immediately begin the walking exercise at a specified intensity for one to five minutes depending on the desired outcome and the recovery from the previous exercise.

Step 19D: Assess the subject physiological state including, but not limited to, heart rate, blood oxygen saturation and current mood.

Step 20A: Reintroduce the subject to a reduced oxygen environment via a mask delivered from a hypoxic generator and continue to monitor the blood oxygen saturation and heart rate.

Step 20B: Reduce and maintain the subject's blood oxygen saturation below 93%, but above 80% for at least 60 seconds to elicit the desired response for the upcoming walking exercise.

Step 20C: The subject must immediately begin the walking exercise at a specified intensity for one to five minutes depending on the desired outcome and the recovery from the previous exercise.

Step 20D: Assess the subject physiological state including, but not limited to, heart rate, blood oxygen saturation and current mood.

Step 21: Reintroduce the subject to a reduced oxygen environment via a mask delivered from a hypoxic generator and continue to monitor the blood oxygen saturation and heart rate.

Step 22: Reduce and maintain the subject's blood oxygen saturation below 93%, but above 80% for at least 60 seconds to elicit the desired response.

Step 23: Refrain from inspiring reduced oxygen until the next exercise session.

### Example 3

Step 1: Assess subject's physiological state including but not limited to heart rate, blood pressure, blood oxygen saturation and current mood.

Step 2A: Introduce the subject to a reduced oxygen environment via a mask delivered from a hypoxic generator and continue to monitor the blood oxygen saturation and heart rate.

Step 2B: Reduce and maintain the subject's blood oxygen saturation below 93%, but above 80% for at least 60 seconds to elicit the desired response for the upcoming 45 degree squat isometric training exercise.

Step 2C: The subject must immediately begin the 45 degree squat isometric training exercise.

Step 2D: Assess the subject physiological state including, but not limited to, heart rate, blood oxygen saturation and current mood.

Step 3A: Reintroduce the subject to a reduced oxygen environment via a mask delivered from a hypoxic generator and continue to monitor the blood oxygen saturation and heart rate.

Step 3B: Reduce and maintain the subject's blood oxygen saturation below 93%, but above 80% for at least 60 seconds to elicit the desired response for the upcoming 45 degree squat isometric training exercise.

Step 3C: The subject must immediately begin the 45 degree squat isometric training exercise.

Step 3D: Assess the subject physiological state including, but not limited to, heart rate, blood oxygen saturation and current mood.

Step 4A: Reintroduce the subject to a reduced oxygen environment via a mask delivered from a hypoxic generator and continue to monitor the blood oxygen saturation and heart rate.

Step 4B: Reduce and maintain the subject's blood oxygen saturation below 93%, but above 80% for at least 60 seconds to elicit the desired response for the upcoming 45 degree squat isometric training exercise.

Step 4C: The subject must immediately begin the 45 degree squat isometric training exercise.

Step 4D: Assess the subject physiological state including, but not limited to, heart rate, blood oxygen saturation and current mood.

Step 5A: Reintroduce the subject to a reduced oxygen environment via a mask delivered from a hypoxic generator and continue to monitor the blood oxygen saturation and heart rate.

Step 5B: Reduce and maintain the subject's blood oxygen saturation below 93%, but above 80% for at least 60 seconds to elicit the desired response for the upcoming horizontal shoulder adduction isometric training exercise.

Step 5C: The subject must immediately begin the horizontal shoulder adduction isometric training exercise.

Step 5D: Assess the subject physiological state including, but not limited to, heart rate, blood oxygen saturation and current mood.

Step 6A: Reintroduce the subject to a reduced oxygen environment via a mask delivered from a hypoxic generator and continue to monitor the blood oxygen saturation and heart rate.

Step 6B: Reduce and maintain the subject's blood oxygen saturation below 93%, but above 80% for at least 60 seconds to elicit the desired response for the upcoming horizontal shoulder adduction isometric training exercise.

Step 6C: The subject must immediately begin the horizontal shoulder adduction isometric training exercise.

Step 6D: Assess the subject physiological state including, but not limited to, heart rate, blood oxygen saturation and current mood.

Step 7A: Reintroduce the subject to a reduced oxygen environment via a mask delivered from a hypoxic generator and continue to monitor the blood oxygen saturation and heart rate.

Step 7B: Reduce and maintain the subject's blood oxygen saturation below 93%, but above 80% for at least 60 seconds to elicit the desired response for the upcoming horizontal shoulder adduction isometric training exercise.

Step 7C: The subject must immediately begin the horizontal shoulder adduction isometric training exercise.

Step 7D: Assess the subject physiological state including, but not limited to, heart rate, blood oxygen saturation and current mood.

Step 8A: Reintroduce the subject to a reduced oxygen environment via a mask delivered from a hypoxic generator and continue to monitor the blood oxygen saturation and heart rate.

Step 8B: Reduce and maintain the subject's blood oxygen saturation below 93%, but above 80% for at least 60 seconds to elicit the desired response for the upcoming scapular retraction isometric training exercise.

Step 8C: The subject must immediately begin the scapular retraction isometric training exercise.

Step 8D: Assess the subject physiological state including, but not limited to, heart rate, blood oxygen saturation and current mood.

Step 9A: Reintroduce the subject to a reduced oxygen environment via a mask delivered from a hypoxic generator and continue to monitor the blood oxygen saturation and heart rate.

Step 9B: Reduce and maintain the subject's blood oxygen saturation below 93%, but above 80% for at least 60 seconds to elicit the desired response for the upcoming scapular retraction isometric training exercise.

Step 9C: The subject must immediately begin the scapular retraction isometric training exercise.

Step 9D: Assess the subject physiological state including, but not limited to, heart rate, blood oxygen saturation and current mood.

Step 10A: Reintroduce the subject to a reduced oxygen environment via a mask delivered from a hypoxic generator and continue to monitor the blood oxygen saturation and heart rate.

Step 10B: Reduce and maintain the subject's blood oxygen saturation below 93%, but above 80% for at least 60 seconds to elicit the desired response for the upcoming scapular retraction isometric training exercise.

Step 10C: The subject must immediately begin the scapular retraction isometric training exercise.

Step 10D: Assess the subject physiological state including, but not limited to, heart rate, blood oxygen saturation and current mood.

Step 11A: Reintroduce the subject to a reduced oxygen environment via a mask delivered from a hypoxic generator and continue to monitor the blood oxygen saturation and heart rate.

Step 11B: Reduce and maintain the subject's blood oxygen saturation below 93%, but above 80% for at least 60 seconds to elicit the desired response for the upcoming shoulder abduction isometric training exercise.

Step 11C: The subject must immediately begin the shoulder abduction isometric training exercise.

Step 11D: Assess the subject physiological state including, but not limited to, heart rate, blood oxygen saturation and current mood.

Step 12A: Reintroduce the subject to a reduced oxygen environment via a mask delivered from a hypoxic generator and continue to monitor the blood oxygen saturation and heart rate.

Step 12B: Reduce and maintain the subject's blood oxygen saturation below 93%, but above 80% for at least 60 seconds to elicit the desired response for the upcoming shoulder abduction isometric training exercise.

Step 12C: The subject must immediately begin the shoulder abduction isometric training exercise.

Step 12D: Assess the subject physiological state including, but not limited to, heart rate, blood oxygen saturation and current mood.

Step 13A: Reintroduce the subject to a reduced oxygen environment via a mask delivered from a hypoxic generator and continue to monitor the blood oxygen saturation and heart rate.

Step 13B: Reduce and maintain the subject's blood oxygen saturation below 93%, but above 80% for at least 60 seconds to elicit the desired response for the upcoming shoulder abduction isometric training exercise.

Step 13C: The subject must immediately begin the shoulder abduction isometric training exercise.

Step 13D: Assess the subject physiological state including, but not limited to, heart rate, blood oxygen saturation and current mood.

Step 14A: Reintroduce the subject to a reduced oxygen environment via a mask delivered from a hypoxic generator and continue to monitor the blood oxygen saturation and heart rate.

Step 14B: Reduce and maintain the subject's blood oxygen saturation below 93%, but above 80% for at least 60 seconds to elicit the desired response for the upcoming shoulder adduction isometric training exercise.

Step 14C: The subject must immediately begin the lat pulldown strength training exercise.

Step 14D: Assess the subject physiological state including, but not limited to, heart rate, blood oxygen saturation and current mood.

Step 15A: Reintroduce the subject to a reduced oxygen environment via a mask delivered from a hypoxic generator and continue to monitor the blood oxygen saturation and heart rate.

Step 15B: Reduce and maintain the subject's blood oxygen saturation below 93%, but above 80% for at least 60 seconds to elicit the desired response for the upcoming shoulder adduction isometric training exercise.

Step 15C: The subject must immediately begin the shoulder adduction isometric training exercise.

Step 15D: Assess the subject physiological state including, but not limited to, heart rate, blood oxygen saturation and current mood.

Step 16A: Reintroduce the subject to a reduced oxygen environment via a mask delivered from a hypoxic generator and continue to monitor the blood oxygen saturation and heart rate.

Step 17B: Reduce and maintain the subject's blood oxygen saturation below 93%, but above 80% for at least 60 seconds to elicit the desired response for the upcoming shoulder adduction isometric training exercise.

Step 17C: The subject must immediately begin the shoulder adduction isometric training exercise.

Step 17D: Assess the subject physiological state including, but not limited to, heart rate, blood oxygen saturation and current mood.

Step 18A: Reintroduce the subject to a reduced oxygen environment via a mask delivered from a hypoxic generator and continue to monitor the blood oxygen saturation and heart rate.

Step 18B: Reduce and maintain the subject's blood oxygen saturation below 93%, but above 80% for at least 60 seconds to elicit the desired response for the upcoming trunk flexion isometric training exercise.

Step 18C: The subject must immediately begin the trunk flexion isometric training exercise.

Step 18D: Assess the subject physiological state including, but not limited to, heart rate, blood oxygen saturation and current mood.

Step 19A: Reintroduce the subject to a reduced oxygen environment via a mask delivered from a hypoxic generator and continue to monitor the blood oxygen saturation and heart rate.

Step 19B: Reduce and maintain the subject's blood oxygen saturation below 93%, but above 80% for at least 60 seconds to elicit the desired response for the upcoming trunk flexion isometric training exercise.

Step 19C: The subject must immediately begin the trunk flexion isometric training exercise.

Step 19D: Assess the subject physiological state including, but not limited to, heart rate, blood oxygen saturation and current mood.

Step 20A: Reintroduce the subject to a reduced oxygen environment via a mask delivered from a hypoxic generator and continue to monitor the blood oxygen saturation and heart rate.

Step 20B: Reduce and maintain the subject's blood oxygen saturation below 93%, but above 80% for at least 60 seconds to elicit the desired response for the upcoming trunk flexion isometric training exercise.

Step 20C: The subject must immediately begin the trunk flexion isometric training exercise.

Step 20D: Assess the subject physiological state including, but not limited to, heart rate, blood oxygen saturation and current mood.

Step 21: Reintroduce the subject to a reduced oxygen environment via a mask delivered from a hypoxic generator and continue to monitor the blood oxygen saturation and heart rate.

Step 22: Reduce and maintain the subject's blood oxygen saturation below 93%, but above 80% for at least 60 seconds to elicit the desired response.

Step 23: Refrain from inspiring reduced oxygen until the next exercise session.

1. A method of a subject inspiring reduced oxygen during the inactive period of a series of activities for physiological benefit, comprising:
a. assessing a subject
b. introducing inspiration of reduced oxygen to said subject
c. maintaining said subject's blood oxygen saturation below a predetermined level for a sufficient amount of time
d. said subject immediately proceeds to predetermined activity
e. assessing said subject
f. repeating b, c, d, e in succession for duration of activity period

2. A method of claim 1 whereby said physiological benefit is a means for improved muscular force output

3. A method of claim 1 whereby said physiological benefit is a means for improved cardiovascular output

4. A method of claim 1 whereby said physiological benefit is a means for improved pulmonary function in volume and rhythm

5. A method of claim 1 whereby said physiological benefit is a means for a multitude of tissues growth, repair, and development

6. A method of claim 1 whereby said physiological benefit is a means for improved vascularity

7. A method of claim 1 whereby said physiological benefit is a means for improved regulation of growth, maintenance, proliferation, and survival of neurons.

8. A method of claim 1 whereby said physiological benefit is a means for improved synaptic strength and excitability of neurons

9. A method of claim 1 whereby said physiological benefit is a means for aiding a multitude of neurodegenerations
